(19) United States
(12) Patent Application Publication
(10) Pub. No.: US 2024/0133879 A1
(43) Pub. Date: Apr. 25, 2024

(54) SMALL MOLECULE SCREENING ASSAY FOR DIGITAL MICROFLUIDIC PLATFORM

(71) Applicant: NICOYA LIFESCIENCES, INC., Kitchener (CA)

(72) Inventors: Theodore WINGER, Durham, NC (US); Chanel LEONG, Ghent (BE)

(21) Appl. No.: 18/262,982

(22) PCT Filed: Jan. 24, 2022
(86) PCT No.: PCT/US2022/013535

§ 371 Date: Jul. 25, 2023

Related U.S. Application Data
(60) Provisional application No. 63/142,179, filed on Jan. 27, 2021.
(60) Provisional application No. 63/228,974, filed on Aug. 3, 2021.

(51) Int. Cl. G01N 33/557; B01L 3/00

(52) U.S. Cl. CPC G01N 33/557; B01L 3/502792

(57) ABSTRACT

Microfluidic devices and methods for assaying molecules in aqueous droplets are provided. The methods include the steps of loading an aqueous sample onto a microfluidic device, dispensing one or more sample droplets into an oil filled droplet operations gap of the microfluidic device and optionally diluting the sample droplet, and transporting a sample droplet to a sensor zone and initiating the assay.

### 1. CROSS REFERENCE TO RELATED APPLICATIONS

This patent application claims the benefit of US Patent App. Nos. 63/142,179 filed Jan. 27, 2021 and 63/228,974 filed Aug. 3, 2021. Both of the above applications are incorporated by reference herein. The entire contents of each of the prior applications are incorporated by reference herein.

### 2. FIELD OF THE INVENTION

The subject matter relates generally to assaying molecules and more particularly to a small molecule screening assay for a digital microfluidic platform.

### 3. BACKGROUND OF THE INVENTION

Small molecule drugs account for a relatively large percentage of therapeutics in the current pharmaceutical market. Accordingly, small molecule drugs also figure prominently in current drug development pipelines. Small molecule drugs may be described as a diverse group of chemical compounds that often have nothing in common other than their small size. For example, the hydrophobicity of small molecule drugs may vary depending on the small molecule.

In development pipelines, various screening platforms may be used to identify and characterize a small molecule compound as a potential therapeutic agent. For example, a surface plasmon resonance (SPR)-based detection assay may be used to screen and identify a small molecule compound as a potential therapeutic agent. In one example, an SPR-based detection assay may be performed on a microfluidic device wherein the small molecule compound is in an aqueous droplet that is surrounded by oil. However, a hydrophobic small molecule may partition from the aqueous droplet into the surrounding oil which may be problematic in a screening assay that is dependent on a known starting concentration of the small molecule candidate. There is a need for methods of preventing a hydrophobic small molecule in an aqueous droplet from partitioning into a surrounding oil phase in a small molecule screening assay performed on a microfluidic device.

### 4. BRIEF DESCRIPTION OF DRAWINGS

FIG. 1 is a cross-sectional view illustrating an example of a portion of a microfluidic device for performing a small molecule binding assay in droplets.

FIG. 2 is a schematic diagram illustrating release of a hydrophobic small molecule guest from a cyclodextrin complex via guest displacement.

FIG. 3 is a flow diagram illustrating an example of a method of screening a hydrophobic small molecule analyte for ligand binding on a microfluidic device wherein the small molecule analyte is immobilized in a cyclodextrin complex.

### 5. SUMMARY OF THE INVENTION

The disclosure provides a method for assaying a small molecule. The method may include providing a droplet actuator where the droplet actuator may include two substrates separated to form a droplet operations gap. The substrates may include electrodes arranged for conducting droplet operations. The droplet operations gap may have an oil filler fluid.

The method may include loading a capture element-small molecule complex aqueous sample onto the droplet actuator. The capture element-small molecule complex may include a capture element and a small molecule analyte forming an inclusion complex.

The method may include dispensing one or more sample droplets from the aqueous sample. Dispensing may be accomplished using droplet operations, such as electrowetting-mediated droplet operations.

The method may optionally include diluting the one or more sample droplets one or more times. For example, diluting may be achieved by using droplet operations to merge the sample droplet with one or more droplets that may include a dilution buffer.

The method may include transporting one of the sample droplets to an assay sensor zone. For example, the transporting may be accomplished using droplet operations, such as electrowetting-mediated droplet operations.

The method may include releasing a small molecule from the capture element-small molecule complex. The method may include conducting an assay using the released small molecule. In some embodiments, the assay may be a binding assay. For example, the assay may include measuring binding kinetics of the released small molecule to a ligand. The method or various steps of the method may be repeated one or more times.

In some embodiments, the capture element may be selected from the group consisting of a cyclodextrin, β-cyclodextrin, carrier bead, liposome, Metal Organic Framework (MOF), metallacage (metal-based supramolecular coordination cage), helicate construct, polymer network, DNA-based construct, and gold-nanoparticle.

In some embodiments, the releasing may include application of light. In some embodiments, the light may be provided via a sensor optical guide.

In some embodiments, the capture element may be a cyclodextrin or a 3-cyclodextrin and the releasing may include introduction of a displacing guest molecule agent.

In some embodiments, the displacing guest molecule agent may be selected from the group consisting of adamantane carboxylate, SDS, a bolaform surfactant, C10-bis(ammonium bromide), C12-bis(ammonium bromide), and combinations thereof.

In some embodiments, the capture element may be a cyclodextrin or a or a β-cyclodextrin and the releasing may include application of light.

In some embodiments, the capture element may be a liposome and the releasing may include application of light.

In some embodiments, the capture element may be selected from the group consisting of MOF, polymer network, DNA-based construct and gold-nanoparticle and the releasing may include application of light.

In some embodiments, the capture element may include derivatization with pendants or within the walls and the releasing may include an ultrafast, chemistry-based guest release triggering mechanism. In some embodiments, the ultrafast, chemistry-based guest release triggering mechanism may include tetrazine-cyclooctene.

In some embodiments, pendants on a capture element may include a functional group which may be attached to the structural portion of the capture element but may not necessarily be part of the main structural portion. For example, the organic ligand subunit of a metallacage may have an arm or pendant which may not participate in covalent coordination to the metal nodes, but may interact with a guest molecule encapsulated inside the cage. Pendants may include photoactive moieties which can undergo light-induced structural transformations; these may include azobenzene, butadienes and spiropyran. When stimulated with light, the change in the pendant's structure (e.g. cis-trans isomerisation in the case of azobenzene, or ring open-close in the case of spiropyran) may facilitate displacement of the small molecule from the capture element (physical displacement and/or change in host-guest enthalpy of adsorption).

In some embodiments, derivatisation of the walls may include constructing a supramolecular cage with conjugated and/or aromatic subunits including phenyl, fused ring polyaromatic hydrocarbons, alkene, alkyne, etc., which form a hydrophobic interior of the cage. Displacement may be achieved by introducing a competing guest which interacts more favourably with the cage than the small molecule. Another mechanism may be to derivatise the walls with acidic/basic groups such as dialkylammonium, anilinium, pyridinium, ureido, etc., such that changes in pH may trigger a change in the host-guest enthalpy of adsorption.

In some embodiments, the capture element may include a carrier bead used to anchor and transport the small molecule. In some embodiments, the small molecule may be anchored to the carrier bead via a cleavable moiety. In some embodiments, the cleavable moiety may include a carboxy group.

In some embodiments, the carrier bead may be a magnetically responsive bead. The carrier bead may include a magnetic core and a porous outer shell, wherein the small molecule may be adsorbed into the outer shell, and wherein the releasing may include an applied alternating magnetic field. In some embodiments, the carrier bead may be suspendable in the aqueous sample. The carrier bead may be an agarose bead. The carrier bead may include a functionalization for binding the small molecule. In some embodiments, the functionalization may include a cyclodextrin or a β-cyclodextrin.

In some embodiments, the oil filler fluid may include a silicone oil. For example, the silicone oil may be a polydimethylsiloxane. The oil filler fluid may include a surfactant. For example, the surfactant may be Span 85.

The disclosure provides a method for assaying molecules in aqueous droplets. The method may include loading an aqueous sample onto a microfluidic device, wherein the aqueous sample may include a molecule to be assayed. The method may include dispensing one or more sample droplets from the sample into a droplet operations gap of the microfluidic device having an oil filler fluid, wherein the oil filler fluid may include a perfluorinated oil. Dispensing may be accomplished using droplet operations, such as electrowetting-mediated droplet operations.

The method may optionally include diluting the sample droplet one or more times. For example, diluting may be accomplished by using droplet operations to merge the sample droplet with one or more droplets that may include a dilution buffer.

The method may include transporting one of the sample droplets to an assay sensor zone. For example, the transporting may be accomplished using droplet operations, such as electrowetting-mediated droplet operations.

The method may include conducting an assay using the sample droplet. In some embodiments, the assay may be a binding assay. For example, the assay may include measuring binding kinetics of the molecule to a ligand. The method or various steps of the method may be repeated one or more times.

In some embodiments, the perfluorinated oil may be selected from the group consisting of GALDEN® HT-170, GALDEN® D, GALDEN® LS/HS, GALDEN® SV, GALDEN® HT low and high boilers series, 3M™ Novec™ Engineered Fluids series, and 3M™ Fluorinert™ Electronic Liquid series. In some embodiments, the filler fluid further may include a combination of the perfluorinated oil and a perfluorinated surfactant. In some embodiments, the perfluorinated surfactant may be selected from the group consisting of Capstone™ FS-83, Capstone™ FS series, 3M™ Fluorosurfactants, and Zonyl® FSO-100. In some embodiments, the combination of the perfluorinated oil and the perfluorinated surfactant may be selected from the group consisting of members of Table 1. In some embodiments, the combination of the perfluorinated oil and the perfluorinated surfactant may be a surfactant saturated oil mixture prepared by mixing the perfluorinated surfactant into the perfluorinated oil until phase separation occurs. In some cases, a combination of a perfluorinated oil and a perfluorinated surfactant may be used to retain a hydrophobic small molecule analyte in an aqueous droplet within the oil filled gap of a microfluidic device.

The disclosure provides a method for assaying molecules in aqueous droplets. The method may include loading an aqueous sample onto a microfluidic device, wherein the aqueous sample may include a molecule to be assayed. The method may include dispensing one or more sample droplets from the sample into a droplet operations gap of the microfluidic device having an oil filler fluid. Dispensing may be accomplished using droplet operations, such as electrowetting-mediated droplet operations. The oil filler fluid may include a combination of a non-fluorinated oil and a fluorinated oil, wherein the combination minimizes partitioning of the molecule into the oil filler fluid.

The method may optionally include diluting the sample droplet one or more times. For example, diluting may be achieved by using droplet operations to merge the sample droplet with one or more droplets that may include a dilution buffer.

The method may include transporting one of the sample droplets to an assay sensor zone. For example, the transporting may be accomplished using droplet operations, such as electrowetting-mediated droplet operations.

The method may include conducting the assay using the sample droplet. In some embodiments, the assay may be a binding assay. For example, the assay may include measuring binding kinetics of the molecule to a ligand. The method or various steps of the method may be repeated one or more times.

In some embodiments, the non-fluorinated oil may be selected from the group consisting of polydimethylsiloxane (PDMS) family of oils; and the fluorinated oil may be selected from the group consisting of GALDEN® HT-170, GALDEN® D, GALDEN® LS/HS, GALDEN® SV, GALDEN® HT low and high boilers series, 3M™ Novec™ Engineered Fluids series, and 3M™ Fluorinert™ Electronic Liquid series.

In some embodiments, the combination of the non-fluorinated oil and the fluorinated oil may prevent a coating on a surface of the droplet operations gap from being dissolved by the fluorinated oil.

In some embodiments, the droplet operations gap may include a physical barrier to minimize contact between the non-fluorinated oil and the fluorinated oil to maintain integrity of the coating on the surface of the droplet operations gap. In some embodiments, the physical barrier may be a wall.

The disclosure provides a method for assaying molecules in aqueous droplets. The method may include loading an aqueous sample onto a microfluidic device, wherein the aqueous sample may include a molecule to be assayed. The method may include dispensing one or more sample droplets from the sample into a droplet operations gap of the microfluidic device. Dispensing may be accomplished using droplet operations, such as electrowetting-mediated droplet operations. The droplet operations gap may include one or more areas having empty air space and one or more areas having an oil filler fluid.

The method may optionally include diluting the sample droplet one or more times. For example, diluting may be achieved by using droplet operations to merge the sample droplet with one or more droplets that may include a dilution buffer.

The method may include transporting one of the sample droplets to an assay sensor zone. For example, the transporting may be accomplished using droplet operations, such as electrowetting-mediated droplet operations.

The method may include conducting an assay using the sample droplet. In some embodiments, the assay may be a binding assay. For example, the assay may include measuring binding kinetics of the molecule to a ligand. The method or various steps of the method may be repeated one or more times.

In some embodiments, the oil filler fluid may include fluorinated oil and the droplet operations gap may include one or more fluorinated oil-free zones.

In some embodiments, a region of the droplet operations gap may include a lower gap height and a second region may include a higher gap height, wherein the higher gap height region may be higher than the lower gap height region. In some embodiments, a volume of the fluorinated oil may be sufficient to fill the lower gap height but not the higher gap height. In some embodiments, the microfluidic device may have a vertical orientation, wherein a region of the droplet operations gap may include the oil filler fluid and another region of the droplet operations gap lacks the oil filler fluid. For example, a bottom portion of the device may include the oil filler fluid and a top portion of the droplet operations gap may be empty of the oil filler fluid.

The disclosure provides a method for assaying molecules in aqueous droplets. The method may include, loading an aqueous sample onto a microfluidic device. The aqueous sample may include one or a class of molecules to be assayed, wherein the aqueous sample may include one or a mixture of surfactants.

The method may include dispensing one or more sample droplets from the sample into a droplet operations gap of the microfluidic device. Dispensing may be accomplished using droplet operations, such as electrowetting-mediated droplet operations. The microfluidic device may include an oil filler fluid, wherein the one or the mixture of surfactants provides anti-partitioning properties to the one or the class of molecules for retention in the aqueous sample.

The method may optionally include diluting the one or more sample droplets one or more times. For example, diluting may be accomplished by using droplet operations to merge the sample droplet with one or more droplets that may include a dilution buffer.

The method may include transporting one of the sample droplets to an assay sensor zone. For example, the transporting may be accomplished using droplet operations, such as electrowetting-mediated droplet operations.

The method may include conducting an assay using the sample droplet. In some embodiments, the assay may be a binding assay. For example, the assay may include measuring binding kinetics of the molecule to a ligand. The method or various steps of the method may be repeated one or more times.

In some embodiments, the one or the class of molecules may include proteins or small molecules.

In some embodiments, a size of the sample droplet ranges from 1X droplet, 2X droplet, . . . nX droplet.

In some embodiments, the one or the mixture of surfactants may include a mixture of one or more non-ionic surfactants and sodium dodecyl sulfate (SDS). In some embodiments, a concentration of the one or the mixture of surfactants may be about a 1.5× critical micelle concentration.

In some embodiments, the oil filler fluid may include a silicone oil. In some embodiments, the oil filler fluid may include a surfactant. In some embodiments, the silicone oil may be a polydimethylsiloxane. In some embodiments, the surfactant may be Span 85.

The disclosure provides a method for assaying molecules in aqueous droplets. The method may include loading an aqueous sample onto a microfluidic device. The aqueous sample may include the molecule to be assayed. The microfluidic device may include a droplet operations gap which may include an oil filler fluid. The method may include dispensing one or more sample droplets from the sample into a liquid delivery system of a fiber. Dispensing may be accomplished using droplet operations, such as electrowetting-mediated droplet operations.

The method may optionally include diluting the sample droplet one or more times. For example, diluting may be accomplished by using droplet operations to merge the sample droplet with one or more droplets that may include a dilution buffer.

The method may include transporting one of the sample droplets via the liquid delivery system to an assay sensor zone. For example, the transporting may be achieved using droplet operations, such as electrowetting-mediated droplet operations. In some embodiments, contact between the sample droplet and the oil filler fluid of the microfluidic device may be substantially avoided.

The method may include conducting an assay using the sample droplet. In some embodiments, the assay may be a binding assay. For example, the assay may include measuring binding kinetics of the molecule to a ligand. The method or various steps of the method may be repeated one or more times.

In some embodiments, the fiber may be a coaxial fiber. In some embodiments, the coaxial fiber may include a central light guide core for delivering a light source surrounded by the liquid delivery system for delivering the one or more sample droplets to the assay sensor zone. In some embodiments, the central light guide core may be an optical guide.

In some embodiments, the liquid delivery system of the fiber may be used to deliver the sample droplet into the droplet operations gap without using electrowetting operations.

In some embodiments, the assay sensor zone may include a surface plasmon resonance (SPR) sensor having the ligand immobilized on the SPR sensor. In some embodiments, the assay sensor zone may include the surface plasmon resonance (SPR) sensor having a protein ligand immobilized on the SPR sensor. In some embodiments, the assay sensor zone may include a modification to provide electrochemical properties to trigger the releasing of the small molecule from the capture element.

In some embodiments, the SPR sensor may be provided on a tip of the coaxial fiber. In some embodiments, the coaxial fiber may include the optical guide that extends into the assay sensor zone. In some embodiments, the optical guide may be a fiber optic light guide. In some embodiments, the tip of the fiber optic light guide may include a modification to provide electrochemical properties to trigger the releasing of the small molecule from the capture element. In some embodiments, the optical guide may be a surface treated fiber optic light guide, wherein a surface treatment of the light guide may provide additional properties to an environment of the sensor. In some embodiments, the optical guide may include a modification to provide electrochemical properties to trigger the releasing of the small molecule from the capture element.

### 6. DETAILED DESCRIPTION OF THE INVENTION

6.1. Small Molecule Screening Assay for Digital Microfluidic Platform

The invention provides a microfluidic device including droplets subject to manipulation by the device wherein the droplet may be an aqueous droplet including a hydrophobic small molecule analyte that may be surrounded by an oil filler fluid in a droplet operations gap of the microfluidic device.

In various aspects, the invention provides methods of retaining a hydrophobic small molecule analyte in an aqueous droplet within the oil filled gap of the microfluidic device.

In one aspect, a method of the invention uses an oil filler fluid including a perfluorinated oil to retain a hydrophobic small molecule analyte in an aqueous droplet within the oil filled gap of a microfluidic device. In some cases, a combination of a perfluorinated oil and a perfluorinated surfactant may be used to retain a hydrophobic small molecule analyte in an aqueous droplet within the oil filled gap of a microfluidic device.

In one aspect, a method of the invention uses an oil filler fluid including a combination of a non-fluorinated oil and a fluorinated oil to retain a hydrophobic small molecule in an aqueous droplet within the oil filled gap of a microfluidic device.

In one aspect, a method of the invention uses a surfactant or a mixture of surfactants to provide universal or substantially universal anti-partitioning properties to the aqueous phase of a droplet including a hydrophobic small molecule.

The invention uses a capture and release system to retain a hydrophobic small molecule analyte in an aqueous droplet within the oil filled gap of a microfluidic device. A capture element may be used to capture a small molecule analyte forming a capture element-small molecule complex. In some aspects the capture element-small molecule complex may be a capture element-small molecule inclusion complex. In other aspects, a small molecule analyte may be released from the capture element for analysis and/or screening. At a point-of-assay, such as at or near a sensor and detection site, the small molecule analyte can be released from the capture element complex for screening in a binding assay.

The capture and release system may make use of a capture element for capture of a hydrophobic small molecule into a capture element complex for processing in a binding assay on a microfluidic device.

In one aspect, a capture element uses a displacement mechanism to release a small molecule analyte from a capture element complex at the point-of-assay (e.g., at or near a sensor and detection site).

A capture element capture and release system may, for example, make use of a light-triggered mechanism to release a small molecule analyte from a capture element complex at the point-of-assay (e.g., at or near a sensor and detection site).

In various aspects, the capture and release system uses a cyclodextrin for capture of a hydrophobic small molecule into a cyclodextrin inclusion complex for processing in a binding assay on a microfluidic device.

In one aspect, a cyclodextrin capture and release system uses a displacement mechanism to release a small molecule analyte from a cyclodextrin complex at the point-of-assay (e.g., at or near a sensor and detection site).

In one aspect, a cyclodextrin capture and release system uses a light-triggered mechanism to release a small molecule analyte from a cyclodextrin complex at the point-of-assay (e.g., at or near a sensor and detection site).

In one aspect, a method of the invention uses a surfactant or mixture of surfactants as an additive in the aqueous droplet phase to retain a hydrophobic small molecule in the aqueous droplet within the oil filled gap of a microfluidic device.

In various aspects, the invention uses carrier beads to anchor and transport a hydrophobic small molecule analyte to a sensor and detection site of a microfluidic device.

In one aspect, the invention uses a fiber (e.g., a coaxial fiber) for delivering an aqueous droplet (solution) containing a hydrophobic small molecule to the sensor and detection site of a microfluidic device.

The methods of the invention include use of a surface plasmon resonance (SPR) sensor with a ligand immobilized thereon for characterizing the affinity and kinetics of a small molecule analyte binding to the ligand.

In one aspect, the sensor may be provided on the tip of a fiber that includes an optical guide that extends into a sensing and detection site of a microfluidic device.

6.1.1. Small Molecule Retaining Systems

6.1.1.1. Perfluorinated Oils and Surfactants

The invention makes use of an oil filler fluid including an oil or mixture of oils, and a surfactant(s) that are selected for retaining a hydrophobic small molecule in an aqueous phase within the oil filled gap of a microfluidic device.

In one aspect, the oil filler fluid may include a perfluorinated oil.

In one aspect, the oil filler fluid may include a mixture of perfluorinated oils and perfluorinated surfactants.

Examples of perfluorinated oils suitable for retaining a hydrophobic small molecule in an aqueous droplet in the oil filled gap of a microfluidic device include but are not limited to: GALDEN® (Solvay) HT-170, GALDEN® D, GALDEN® LS/HS, GALDEN® SV, GALDEN® HT low and high boilers series (available from Solvay, Inc.), 3M™ Novec™ Engineered Fluids series (available from 3M, Inc.), and 3M™ Fluorinert Electronic Liquid series (available from 3M, Inc.).

Examples of perfluorinated surfactants suitable for retaining a hydrophobic small molecule in an aqueous droplet in the oil filled gap of a microfluidic device include: Capstone™ FS-83 or others of the Capstone™ FS series (available from Chemours, Inc.), and 3M™ Fluorosurfactants (available from 3M, Inc.). Other examples of fluorinated surfactants include Zonyl® FSO-100 (from Sigma-Aldrich, Inc.).

In one aspect of the invention, a method is provided for assaying molecules in aqueous droplets. The method includes, but is not limited to, the steps of:
(a) loading an aqueous sample including a molecule to be assayed onto a microfluidic device that contains an oil filler fluid, wherein the oil filler fluid is a combination of a perfluorinated oil and a perfluorinated surfactant;
(b) dispensing one or more sample droplets from the sample into a droplet operations gap of the microfluidic device, wherein dispensing may be accomplished using droplet operations, such as electrowetting-mediated droplet operations;
(c) optionally, diluting the sample droplet one or more times to yield a diluted sample droplet, wherein diluting may be achieved by using droplet operations to merge the sample droplet with one or more droplets that may include a dilution buffer;
(d) transporting the sample droplet or diluted sample droplet to an assay sensor zone of the microfluidic device, wherein the transporting may be accomplished using droplet operations, such as electrowetting-mediated droplet operations;
(e) initiating the assay; and wherein the steps of transporting a sample droplet to the sensor zone and initiating the assay can be repeated one or more times.

In one aspect, the assay is a binding assay wherein the binding kinetics of the molecule to a ligand are measured.

In one aspect, the perfluorinated oil is selected from the group consisting of GALDEN® HT-170, GALDEN® D, GALDEN® LS/HS, GALDEN® SV, GALDEN® HT low and high boilers series, 3M™ Novec™ Engineered Fluids series, and 3M™ Fluorinert Electronic Liquid series.

In one aspect, the perfluorinated surfactant is selected from the group consisting of Capstone™ FS-83, Capstone™ FS series, 3M™ Fluorosurfactants, and Zonyl® FSO-100.

In one aspect, the combination of the perfluorinated oil and the perfluorinated surfactant is selected from the group consisting of members of Table 1.

In one aspect, the combination of a perfluorinated oil and a perfluorinated surfactant is a surfactant saturated oil mixture prepared by mixing an appropriate amount of perfluorinated surfactant into a perfluorinated oil to achieve the desired interfacial surface energy with the aqueous droplet.

In one aspect, the assay sensor zone may include a surface plasmon resonance (SPR) sensor having a ligand immobilized on the SPR sensor.

In one aspect, the ligand is a protein.

6.1.1.2. Non-fluorinated and Fluorinated Oils

In one aspect, the oil filler fluid may include a mixture of non-fluorinated and fluorinated oils. The mixture of non-fluorinated and fluorinated oils is selected for minimizing the rate of partitioning of a hydrophobic small molecule in an aqueous droplet into the oil filler fluid, while maintaining the integrity of a surface coating (e.g., CYTOP) during the assay process. Examples of non-fluorinated oils include oils from the polydimethylsiloxane (PDMS) family (from Gelest, Inc.). Examples of fluorinated oils include oils from the GALDEN® series (from Solvay, Inc) or the Novec™ series (from 3M, Inc.).

In one example, the mixture of non-fluorinated and fluorinated oils is selected to be compatible with a coating (e.g., a CYTOP coating) on the surface of the droplet operations gap of a microfluidic device. The use of a mixture of fluorinated and non-fluorinated oils would prevent a coating such as CYTOP from being dissolved by the fluorinated oil.

In one aspect, one or more areas in the droplet operations gap of a digital microfluidic device may be empty of oil (e.g., air spaces) and the other areas may contain an oil filler fluid. For example, one half of the droplet operations gap may be filled with an oil filler fluid and the other half of the device does not contain an oil filler fluid. In this example, electrowetting for droplet operations is performed in air areas without an oil filler fluid (i.e., electrowetting is performed in air) and in areas containing an oil filler fluid. The use of fluorinated oil-free zones in the droplet operations gap of a digital microfluidic device enables the use of a surface coating such as CYTOP.

In one example, a region of the droplet operations gap of a digital microfluidic device has a lower gap height and a region of the droplet operations gap has a higher gap height, wherein the higher gap height region is higher than the lower gap height region. A volume of an oil filler fluid (e.g., a fluorinated oil) may be used, wherein the volume used is sufficient to fill the lower gap height region of the droplet operations gap, but is not sufficient to fill the region of the droplet operations gap having a higher gap height. The use of fluorinated oil-free zones in the droplet operations gap of a digital microfluidic device enables the use of a surface coating such as CYTOP.

In one aspect, the invention makes use of a digital microfluidic device having a vertical orientation, wherein only a region of the device contains an oil filler fluid (e.g., a fluorinated oil). In one example, the bottom region of the vertical digital microfluidic device contains an oil filler fluid and the top region of the device lacks the oil filler fluid. The use of fluorinated oil-free zones in the droplet operations gap of a digital microfluidic device enables the use of a surface coating such as CYTOP.

In one aspect of the invention, a method is provided for assaying molecules in aqueous droplets. The method includes, but is not limited to, the steps of:
(a) loading an aqueous sample including a molecule to be assayed onto a microfluidic device that contains an oil filler fluid, wherein the oil filler fluid is a combination of a non-fluorinated oil and a fluorinated oil that minimizes partitioning of the molecule into the oil filler fluid;
(b) dispensing one or more sample droplets from the sample into a droplet operations gap of the microfluidic device, wherein dispensing is accomplished using droplet operations, such as electrowetting-mediated droplet operations;
(c) optionally, diluting the sample droplet one or more times to yield a diluted sample droplet, wherein diluting may be achieved by using droplet operations to merge the sample droplet with one or more droplets that may include a dilution buffer;
(d) transporting the sample droplet or diluted sample droplet to an assay sensor zone of the microfluidic device, wherein the transporting may be accomplished using droplet operations, such as electrowetting-mediated droplet operations;
(e) initiating the assay; and wherein the steps of transporting a sample droplet to the sensor zone and initiating the assay can be repeated one or more times.

In one aspect, the non-fluorinated oil is selected from the group consisting of polydimethylsiloxane (PDMS) family of oils; and the fluorinated oil is selected from the group consisting of GALDEN® HT-170, GALDEN® D, GALDEN® LS/HS, GALDEN® SV, GALDEN® HT low and high boilers series, 3M™ Novec™ Engineered Fluids series, and 3M™ Fluorinert Electronic Liquid series.

In one aspect, the combination of the non-fluorinated oil and the fluorinated oil prevents a coating on the surface of the droplet operations gap from being dissolved by the fluorinated oil.

In one aspect, the droplet operations gap may include a physical barrier to minimize contact between the non-fluorinated oil and the fluorinated oil to maintain integrity of a coating on the surface of the droplet operations gap. In one aspect, the physical barrier is a wall.

In one aspect, one or more physical barriers (e.g., a wall) may be used to separate and minimize contact between a fluorinated oil and a non-fluorinated oil in the droplet operations gap of a digital microfluidic device. The use of a physical barrier(s) to minimize contact between a fluorinated oil and a non-fluorinated oil may be used to maintain the integrity of a coating such as CYTOP on the surface of the droplet operations gap of a microfluidic device.

In one aspect, a method is provided for assaying molecules in aqueous droplets, the method including, but not limited to, the steps of:
(a) loading an aqueous sample including a molecule to be assayed onto a microfluidic device that includes a droplet operations gap having one or more areas having empty air space and one or more areas having an oil filler fluid;
(b) dispensing one or more sample droplets from the sample into a droplet operations gap of the microfluidic device, wherein dispensing may be accomplished using droplet operations, such as electrowetting-mediated droplet operations;
(c) optionally, diluting the sample droplet one or more times to yield a diluted sample droplet, wherein diluting may be achieved by using droplet operations to merge the sample droplet with one or more droplets that may include a dilution buffer;
(d) transporting the sample droplet or diluted sample droplet to an assay sensor zone of the microfluidic device, wherein the transporting may be accomplished using droplet operations, such as electrowetting-mediated droplet operations;
(e) initiating the assay; and wherein the steps of transporting a sample droplet to the sensor zone and initiating the assay can be repeated one or more times.

In one aspect, the oil filler fluid may include fluorinated oil and the droplet operations gap may include one or more fluorinated oil-free zones.

In one aspect, a region of the droplet operations gap may include a lower gap height and a second region may include a higher gap height, and a volume of the fluorinated oil is sufficient to fill the lower gap height but not the higher gap height.

In one aspect, the digital microfluidic device has a vertical orientation, wherein only a region of the device may include the oil filler fluid.

In one aspect, a bottom region of the device may include the oil filler fluid and a top region of the device lacks the oil filler fluid.

In one aspect, the assay is a binding assay that includes measuring binding kinetics of the molecule to a ligand.

In one aspect, the assay sensor zone may include a surface plasmon resonance (SPR) sensor having a ligand immobilized on the SPR sensor.

In one aspect, the ligand is a protein.

6.1.1.3. Aqueous Phase Surfactants

The invention makes use of a surfactant or a mixture of surfactants to provide universal or substantially universal anti-partitioning properties to the aqueous phase of a droplet including a hydrophobic small molecule.

In one aspect, a mixture of surfactants is selected to provide anti-partitioning properties to a certain class of drugs (e.g., proteins or small molecules), wherein the properties of the class of drugs are known. A screening strategy using a set of small molecule drugs with known properties (e.g., hydrophobicities ranging from log P values −10 to +10) may be used to identify a surfactant or mixture of surfactants that provide anti-partitioning properties. The screening strategy may include, but is not limited to, parameters such as compatibility with the electrowetting environment of the digital microfluidic device, concentrations of the hydrophobic small molecules in an aqueous droplet, and/or droplet size (e.g., 1X droplet, 2X droplet, . . . , nX droplet).

In one example, a mixture of a non-ionic surfactant(s) and SDS may be used to provide universal or substantially universal anti-partitioning properties to the aqueous phase of a droplet including a hydrophobic small molecule. In this example, SDS is used to stop a hydrophobic small molecule from partitioning from the aqueous droplet into the oil filler fluid and a non-ionic surfactant(s) is used to re-nature any SDS-mediated denaturation of biomolecules (e.g., proteins).

In one aspect, a method is provided for assaying molecules in aqueous droplets, the method including, but not limited to, the steps of:
(a) loading an aqueous sample including a molecule or a class of molecules to be assayed and one or a mixture of surfactants having anti-partitioning properties onto a microfluidic device that includes a droplet operations gap having an oil filler fluid;
(b) dispensing one or more sample droplets from the sample into a droplet operations gap of the microfluidic device, wherein dispensing may be accomplished using droplet operations, such as electrowetting-mediated droplet operations;
(c) optionally, diluting the sample droplet one or more times to yield a diluted sample droplet, wherein diluting may be achieved by using droplet operations to merge the sample droplet with one or more droplets that may include a dilution buffer;
(d) transporting the sample droplet or diluted sample droplet to an assay sensor zone of the microfluidic device, wherein the transporting may be accomplished using droplet operations, such as electrowetting-mediated droplet operations;
(e) initiating the assay; and wherein the steps of transporting a sample droplet to the sensor zone and initiating the assay can be repeated one or more times.

In one aspect, the assay is a binding assay and binding kinetics of the molecule to a ligand are measured.

In one aspect, the one or a class of molecules may include proteins or small molecules.

In one aspect, the size of the sample droplet ranges from 1X droplet, 2X droplet, . . . nX droplet.

In one aspect, the one or a mixture of surfactants may include a mixture of one or more non-ionic surfactants and a sodium dodecyl sulfate (SDS).

In one aspect, the concentration of the one or a mixture of surfactants is about a 1.5X critical micelle concentration.

In one aspect, the oil filler fluid may include PDMS and 0.01% Span 85.

In one aspect, the assay sensor zone may include a surface plasmon resonance (SPR) sensor having a ligand immobilized on the SPR sensor.

In one aspect, the ligand is a protein.

6.1.1.4. Small Molecule Capture and Release Mechanism

The invention makes use of a capture and release mechanism for retaining a hydrophobic small molecule in an aqueous phase within the oil filled gap of a microfluidic device. In various aspects a capture or cage element is used to sequester a small molecule in an inclusion complex and then a release mechanism is used to release the hydrophobic small molecule at the point-of-assay (e.g., at a sensor or detector site). The capture or cage element may be selected based on the mechanism of release of a hydrophobic small molecule from the inclusion complex.

In one aspect, a capture and release mechanism uses a displacing guest molecule as a releasing agent.

In one example, the capture or cage element may be a cyclodextrin, such as β-cyclodextrin, and the releasing agent may be a displacing guest molecule such as adamantane carboxylate, SDS, and/or a bolaform surfactant (e.g., C10- or C12-bis(ammonium bromide)).

In one aspect, a capture and release mechanism uses light as a releasing agent. The light may, for example, be provided via a sensor (SPR) optical guide.

In one example, the capture or cage element may be a member of the cyclodextrin family and light is used to trigger release of the small molecule by opening a passage/door in the wall of the capture or cage element (or remove a blockage from a pore in said element, e.g., blocking access to the inner core of a cyclodextrin ring).

In one example, the capture or cage element may be liposomes and light (e.g., near-infrared) is used to trigger release of the small molecule by increasing the porosity of the capture or cage element.

In one example, the capture or cage element may be a Metal Organic Framework (MOF), metallacage (metal-based supramolecular coordination cage) and/or helicate construct and light is used to trigger release of the small molecule by changing the chemical nature of the inner walls of the capture or cage element, thereby releasing the small molecule for diffusion into the bulk of the aqueous phase.

In another example, the capture or cage element may be a MOF, a cyclodextrin, a polymer network, a DNA-based construct, or a liposome and light is used to trigger release of the small molecule by disintegrating the cage element into soluble subunits.

In one example, the capture or cage element may be gold-nanoparticles (“nanobubbles”) that are designed to break up in the evanescence field generated at the gold surface by incidence of light.

Similar cage constructs as the ones described above could be derivatized appropriately with pendants or within their walls in order to enable the use of ultrafast, chemistry-based guest release triggering mechanisms such as the tetrazine-cyclooctene based scheme (see Carlson et al., J Amer Chem Soc (2018) 140, 3603-3612, which is incorporated herein by reference in its entirety).

In some embodiments, pendants on a capture element may include a functional group which may be attached to the structural portion of the capture element but may not necessarily be part of the main structural portion. For example, the organic ligand subunit of a metallacage may have an arm or pendant which may not participate in covalent coordination to the metal nodes, but may interact with a guest molecule encapsulated inside the cage. Pendants may include photoactive moieties which can undergo light-induced structural transformations; these may include azobenzene, butadienes and spiropyran. When stimulated with light, the change in the pendant's structure (e.g. cis-trans isomerisation in the case of azobenzene, or ring open-close in the case of spiropyran) may facilitate displacement of the small molecule from the capture element (physical displacement and/or change in host-guest enthalpy of adsorption).

In some embodiments, derivatisation of the walls may include constructing a supramolecular cage with conjugated and/or aromatic subunits including phenyl, fused ring polyaromatic hydrocarbons, alkene, alkyne, etc., which form a hydrophobic interior of the cage. Displacement may be achieved by introducing a competing guest which interacts more favourably with the cage than the small molecule. Another mechanism may be to derivatise the walls with acidic/basic groups such as dialkylammonium, anilinium, pyridinium, ureido, etc., such that changes in pH may trigger a change in the host-guest enthalpy of adsorption.

In various aspects, the invention uses carrier beads as a capture element to anchor and transport a hydrophobic small molecule analyte to a sensor and detection site of a microfluidic device. For example, a carrier bead may be used to anchor and transport a certain set of hydrophobic small molecule analytes, wherein the set of small molecule analytes are suitable for performing a modification that may then be used to anchor the analyte to a carrier bead without interfering with the analyte-ligand binding reaction.

In one example, the modification is a cleavable moiety that is used to anchor a small molecule analyte to a carrier bead. At a sensing and detection zone of a microfluidic device, the cleavable moiety may be cleaved thereby releasing the small molecule analyte for screening in a binding assay.

In one example, the cleavable moiety is a carboxy group.

In one example, a suitable class of hydrophobic small molecule analytes are characterized by a “handle” or region that is amenable to modification without affecting the binding of the analyte to a target ligand.

In one example a carrier bead may be a magnetically responsive bead.

In one example a carrier bead may be a porous magnetically-responsive bead, that includes a magnetic core (e.g., iron oxide) and porous outer shell (e.g., silica, polymer), which may adsorb a small molecule into the porous outer shell. An applied alternating magnetic field may, for example, be used to trigger the release of the small molecule by heat dissipation.

In another example, a carrier bead may be a bead that is suspendable in an aqueous environment and does not partition into the oil filler fluid of a digital microfluidic device. In one example, the carrier bead is an agarose bead.

In another example, the carrier bead includes a functionalization for binding a small molecule analyte to the carrier bead. In one example, the functionalization is a cyclodextrin. In one example the carrier bead may be functionalized according to the method of Arya et al. (Arya et al., ACS Appl. Mater. Interfaces 2017, 9, 37238-37245).

In one aspect, a method is provided for assaying molecules in aqueous droplets, the method including, but not limited to, the steps of:
(a) loading a capture element-small molecule complex aqueous sample to be assayed onto a microfluidic device that includes a droplet operations gap having an oil filler fluid;
(b) dispensing one or more sample droplets from the sample into a droplet operations gap of the microfluidic device, wherein dispensing may be accomplished using droplet operations, such as electrowetting-mediated droplet operations;
(c) optionally, diluting the sample droplet one or more times to yield a diluted sample droplet, wherein diluting may be achieved by using droplet operations to merge the sample droplet with one or more droplets that may include a dilution buffer;
(d) transporting the sample droplet or diluted sample droplet to an assay sensor zone of the microfluidic device, wherein the transporting may be accomplished using droplet operations, such as electrowetting-mediated droplet operations;
(e) releasing the small molecule from the capture element-small molecule complex to initiate the assay; and wherein the steps of transporting a sample droplet to the sensor zone and initiating the assay can be repeated one or more times.

In one aspect, the assay is a binding assay and binding kinetics of the released small molecule to a ligand are measured.

In one aspect, the capture element is selected from the group consisting of a cyclodextrin, β-cyclodextrin, carrier bead, liposome, Metal Organic Framework (MOF), metallacage (metal-based supramolecular coordination cage), helicate construct, polymer network, DNA-based construct, and gold-nanoparticle.

In one aspect, the releasing may include application of light.

In one aspect, the light is provided via a sensor optical guide.

In one aspect, the capture element is a cyclodextrin or a β-cyclodextrin and the releasing may include introduction of a displacing guest molecule agent.

In one aspect, the displacing guest molecule agent is selected from the group consisting of adamantane carboxylate, SDS, bolaform surfactant, C10-bis(ammonium bromide), C12-bis(ammonium bromide), and combinations thereof.

In one aspect, the capture element is a cyclodextrin or a β-cyclodextrin and the releasing may include application of light.

In one aspect, the capture element is a liposome and the releasing may include application of light.

In one aspect, the capture element is selected from the group consisting of MOF, polymer network, DNA-based construct and gold-nanoparticle and the releasing may include application of light.

In one aspect, the capture element may include derivatization with pendants or within the walls and the releasing may include an ultrafast, chemistry-based guest release triggering mechanism.

In one aspect, the ultrafast, chemistry-based guest release triggering mechanism may include tetrazine-cyclooctene.

In one aspect, pendants on a capture element may include a functional group which may be attached to the structural portion of the capture element but may not necessarily be part of the main structural portion.

In one aspect, the organic ligand subunit of a metallacage may have an arm or pendant which may not participate in covalent coordination to the metal nodes, but may interact with a guest molecule encapsulated inside the cage.

In one aspect, pendants may include photoactive moieties which may undergo light-induced structural transformations; the photoactive moieties may include azobenzene, butadienes and spiropyran.

In one aspect, when stimulated with light, the change in the pendant's structure (e.g. cis-trans isomerisation in the case of azobenzene, or ring open-close in the case of spiropyran) may facilitate displacement of the small molecule from the capture element.

In one aspect, the displacement may be a physical displacement and/or change in host-guest enthalpy of adsorption.

In one aspect, derivatisation of the walls may include constructing a supramolecular cage with conjugated and/or aromatic subunits including phenyl, fused ring polyaromatic hydrocarbons, alkene, alkyne, etc., which form a hydrophobic interior of the cage.

In one aspect, displacement may be achieved by introducing a competing guest moiety which interacts more favourably with the cage than the small molecule.

In one aspect, another mechanism to derivatise the walls may be by attaching acidic/basic groups such as dialkylammonium, anilinium, pyridinium, ureido, etc., such that changes in pH may trigger a change in the host-guest enthalpy of adsorption.

In one aspect, the capture element may include a carrier bead used to anchor and transport the small molecule.

In one aspect, the small molecule is anchored to the carrier bead via a cleavable moiety.

In one aspect, the cleavable moiety may include a carboxy group.

In one aspect, the carrier bead is a magnetically responsive bead.

In one aspect, the carrier bead may include a magnetic core and a porous outer shell, and the small molecule is adsorbed into the outer shell. The releasing may include an applied alternating magnetic field.

In one aspect, the carrier bead is suspendable in the aqueous sample.

In one aspect, the carrier bead is an agarose bead.

In one aspect, the carrier bead may include a functionalization for binding the small molecule.

In one aspect, the functionalization may include a cyclodextrin.

In one aspect, the oil filler fluid may include PDMS and 0.01% Span 85.

In one aspect, the assay sensor zone may include a surface plasmon resonance (SPR) sensor having a ligand immobilized on the SPR sensor.

In one aspect, the ligand is a protein.

6.1.2. Coaxial Fiber

In one aspect, the invention uses a fiber (e.g., a coaxial fiber) for delivering an aqueous droplet (solution) containing a hydrophobic small molecule to the sensor and detection site of a microfluidic device. Because the aqueous droplet (solution) is delivered via the fiber, contact between the hydrophobic small molecule containing droplet and an oil filler fluid is substantially avoided.

In one example, the fiber is a coaxial fiber including a central light guide core for delivering a light source surrounded by a liquid delivery system (e.g., sheath) for delivering the aqueous droplet (solution). The fiber may, for example, be an optical guide fiber for delivering light to the detection zone of a digital microfluidic device.

In another example, the fiber may be used to deliver a liquid material into the droplet operations gap of a digital microfluidic device without the use of electrowetting operations.
(a) loading an aqueous sample including a molecule to be assayed onto a microfluidic device that includes a droplet operations gap having an oil filler fluid;
(b) dispensing one or more sample droplets from the sample into a delivery system of a fiber for delivery of the sample droplets to an assay sensor zone, wherein dispensing may be accomplished using droplet operations, such as electrowetting-mediated droplet operations;
(c) optionally, diluting the sample droplet one or more times to yield a diluted sample droplet, wherein diluting may be achieved by using droplet operations to merge the sample droplet with one or more droplets that may include a dilution buffer;
(d) delivering the sample droplet or diluted sample droplet via the delivery system to the assay sensor zone of the microfluidic device and initiating the assay, such that contact between the sample droplet and the oil filler fluid of the microfluidic device is substantially avoided; and wherein the steps of delivery and assay initiation can be repeated for one or more sample droplets.

In one aspect, the assay is a binding assay and binding kinetics of the molecule to a ligand are measured.

In one aspect, the fiber is a coaxial fiber.

In one aspect, the coaxial fiber has a central light guide core for delivering a light source surrounded by the liquid delivery system for delivering the sample droplets to the assay sensor zone.

In one aspect, the central light guide core is an optical guide fiber.

In one aspect, the liquid delivery system of the fiber is used to deliver the sample droplet into the droplet operations gap without the use of electrowetting operations.

In one aspect, the assay sensor zone may include a surface plasmon resonance (SPR) sensor having a ligand immobilized on the SPR sensor.

In one aspect, the ligand is a protein.

6.1.3. SPR Sensor

The methods of the invention use a surface plasmon resonance (SPR) sensor with a ligand immobilized thereon for characterizing the affinity and kinetics of a small molecule analyte binding to the ligand. In one example, the ligand is a protein. The sensor may be provided on the tip of a fiber that includes an optical guide that extends into a sensing and detection site of a microfluidic device.

In one example, the optical guide is a fiber optic light guide.

In another example, the optical guide is a surface treated fiber optic light guide, wherein the surface treatment of the light guide provides additional properties to the sensor environment.

In another example, a light guide may be modified to provide certain electrochemical properties in the sensing and detection site of a microfluidic device, wherein the electrochemical properties may be used to trigger release of a small molecule from a capture or cage element. For example, the tip of a fiber optic light guide may be modified to provide electrochemical properties to the sensor environment (i.e., the sensing and detection zone of the digital microfluidic device). In yet another example, the sensing and detection zone of a digital microfluidic device in the area of the sensor may be modified to provide electrochemical properties.

In one aspect, the assay sensor zone may include a surface plasmon resonance (SPR) sensor having a ligand immobilized on the SPR sensor.

In one aspect, the assay sensor zone may include a surface plasmon resonance (SPR) sensor having a protein ligand immobilized on the SPR sensor.

In one aspect, the assay sensor zone may include a modification to provide electrochemical properties to trigger the releasing of the small molecule from the capture element.

In one aspect, the SPR sensor is provided on the tip of a fiber including an optical guide that extends into the assay sensor zone.

In one aspect, the optical guide is a fiber optic light guide.

In one aspect, a tip of the fiber optic light guide may include a modification to provide electrochemical properties to trigger the releasing of the small molecule from the capture element.

In one aspect, the optical guide is a surface treated fiber optic light guide, wherein the surface treatment of the light guide provides additional properties to an environment of the sensor.

In one aspect, the optical guide may include a modification to provide electrochemical properties to trigger the releasing of the small molecule from the capture element.

6.1.4. Microfluidic Device and Methods

6.1.4.1. Microfluidic Device and Small Molecule Screening Assays

The invention provides a microfluidic device including droplets subject to manipulation by the device wherein the droplets may include a hydrophobic small molecule for screening in a binding assay.

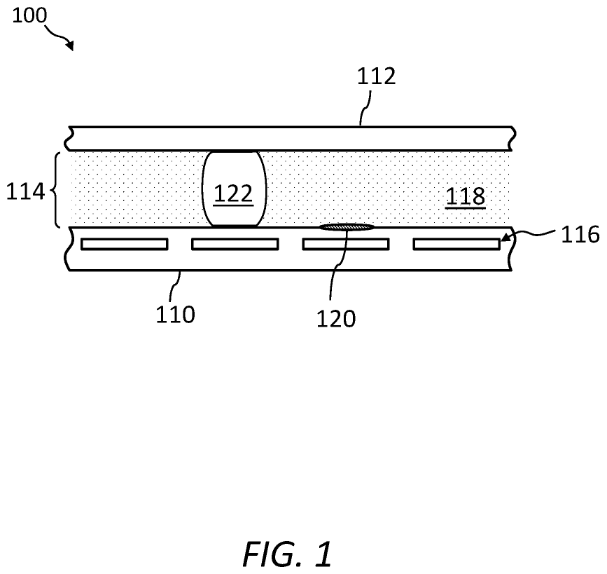

FIG. 1 is a cross-sectional view illustrating an example of a region of a microfluidic device 100 for performing a small molecule binding assay in droplets. Microfluidic device 100 includes a bottom substrate 110 and a top substrate 112 separated by a gap 114. A set of droplet operations electrodes 116, e.g., electrowetting electrodes, are arranged, for example, on bottom substrate 110. The droplet operations electrodes 116 are arranged for conducting droplet operations, such as droplet loading, dispensing, splitting, transporting, merging, and mixing. Gap 114 is filled with an oil filler fluid 118. Oil filler fluid 118 may, for example, be a low-viscosity oil, such as silicone oil.

Microfluidic device 100 further includes a sensor 120. Sensor 120 is arranged in proximity to a certain droplet operations electrode 116 that is positioned in proximity to a detection site (not shown). Sensor 120 may, for example, be a surface plasmon resonance (SPR) sensor with a ligand immobilized thereon for characterizing the affinity and kinetics of a small molecule analyte binding to the ligand. In one example the ligand for binding is a protein. In one example, sensor 120 may be provided on the tip of a fiber as an optical guide, that is positioned in proximity to the detection site of the microfluidic device.

An aqueous droplet 122 may be present in gap 114 of microfluidic device 100. In one example, droplet 122 is a droplet of a small molecule sample to be evaluated for binding to a specific ligand. In another example, droplet 122 is a reagent droplet for conducting a binding assay. Oil filler fluid 118 fills gap 114 and surrounds droplet 122.

In some embodiments of the invention, droplet 122 may include a surfactant that is suitable for retaining a hydrophobic small molecule in the aqueous phase droplet.

In one aspect, the invention uses a capture or cage element to sequester a hydrophobic small molecule in an inclusion complex and a displacing guest molecule as a releasing agent.

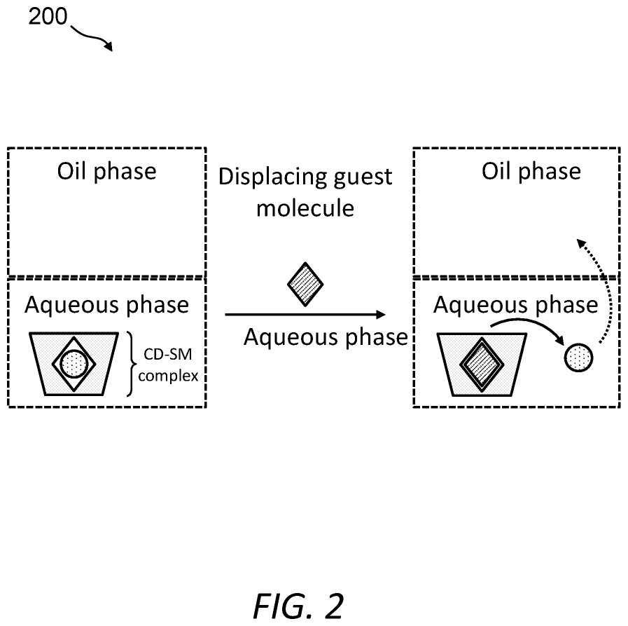

FIG. 2 is a schematic diagram 200 illustrating release of a hydrophobic small molecule guest from a cyclodextrin complex via guest displacement. In this example, the cyclodextrin-small molecule complex (CD-SM) is in an aqueous phase topped by an organic (oil) phase. The CD-SM complex retains a hydrophobic small molecule guest in the aqueous phase and may be used to substantially reduce partitioning of the hydrophobic small molecule into the oil phase. The small molecule guest in the CD-SM complex is released by the addition of a displacing guest molecule that has a higher affinity toward cyclodextrin than the small molecule guest. Upon release from the cyclodextrin complex, the small molecule is free to diffuse from the aqueous phase into the organic (oil) phase.

In one aspect, a method of the invention uses cyclodextrin (CD) to immobilize a small molecule (SM) analyte in a cyclodextrin inclusion complex (CD-SM complex) in an aqueous droplet within the oil filled gap of a microfluidic device. At a point-of-assay, such as at or near a sensor or detection site, the small molecule analyte can be released from the CD-SM complex for screening in a binding assay.

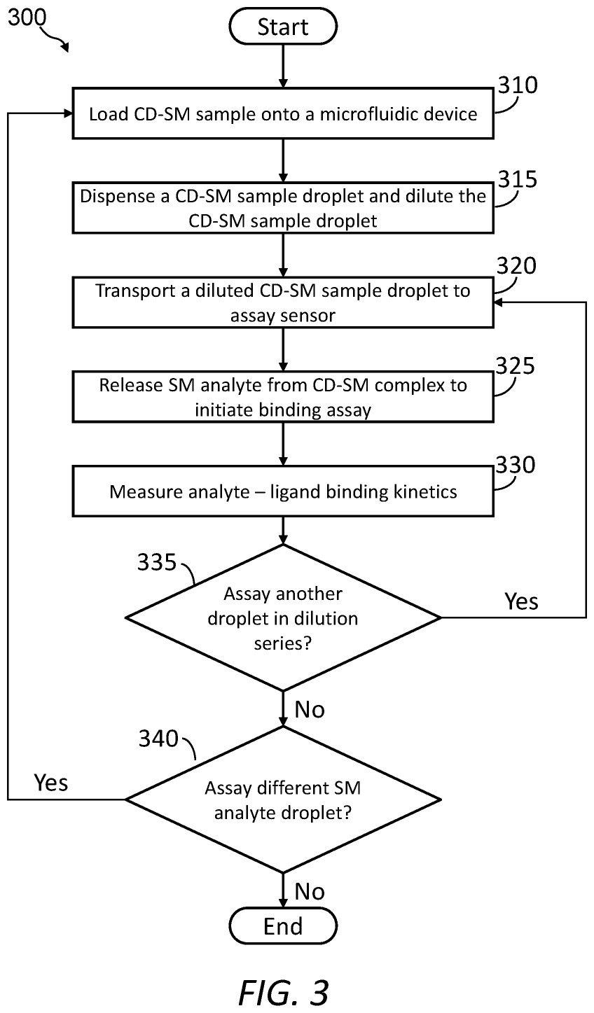

FIG. 3 is a flow diagram illustrating an example of a method 300 of screening a hydrophobic small molecule analyte for ligand binding on a microfluidic device wherein the small molecule analyte is immobilized in a cyclodextrin complex. Method 300 may include any or all of the following steps as well as additional unspecified steps.

At a step 310, a CD-SM sample is loaded onto a microfluidic device. For example, a CD-SM sample in an aqueous buffer solution is loaded into a sample reservoir of the microfluidic device. In one example, the initial concentration of the CD-SM complex in the sample is relatively high, thereby facilitating dilution of the small molecule sample for assaying at two or more different concentrations.

At a step 315, a CD-SM sample droplet is dispensed and diluted. For example, the CD-SM sample droplet may be combined with a diluent droplet to yield a diluted CD-SM sample droplet. The dilution process may be repeated any number of times to yield a series of diluted CD-SM sample droplets. In one example, the dilution process is repeated 3 times to yield 3 serially diluted concentrations of the CD-SM sample. In another example, the dilution process is repeated 5 times to yield 5 serially diluted concentrations of the CD-SM sample. In yet another example, the dilution process is repeated 7 times to yield 7 serially diluted concentrations of the CD-SM sample.

At a step 320, a diluted CD-SM sample droplet is transported to an assay sensor and detection site. In one example, the diluted CD-SM sample droplet is transported to a droplet operations electrode near the assay sensor and detection site. In another example, the diluted CD-SM sample droplet is transported to a droplet operations electrode at the assay sensor and detection site.

At a step 325, the SM analyte is released from the CD-SM complex in the diluted CD-SM sample droplet to initiate the binding assay. In one example, a displacement reagent droplet is combined with the diluted CD-SM sample droplet. The displacement reagent droplet includes a displacing guest molecule with a higher affinity for binding cyclodextrin than the SM analyte, thereby readily displacing the SM analyte from the CD complex.

In one example, the diluted CD-SM sample droplet is combined with the displacing reagent droplet near the sensor site (e.g., at a droplet operations electrode that is adjacent to the sensor site).

In another example, the diluted CD-SM sample droplet is combined with the displacing reagent droplet at the sensor site (i.e., at a droplet operations electrode that is at the sensor site). Because the SM analyte is released at or near the sensor site, the gradient of SM analyte concentration is greatest at the sensor surface, thereby enabling SM detection at the sensor surface.

In another example, a light-triggered mechanism is used to release a SM analyte from the CD complex. The light trigger may, for example, be provided through the sensor optical guide (e.g., a fiber or other).

At a step 330, the analyte-ligand binding kinetics are measured.

At a decision step 335, it is determined whether to assay another droplet in the dilution series. If yes, then method 300 returns to step 320. If no, then method 300 proceeds to a step 340.

At a decision step 340, it is determined whether to assay a different SM analyte. If yes, then method 300 returns to step 310. If no, then method 300 ends.

In some embodiments, a recapture step may be used to recapture any remaining small molecule analyte in the aqueous droplet after the binding response has been measured. The recapture step may be used to prevent diffusion of the released small molecule analyte out of the aqueous droplet and into the surrounding oil filler fluid. The recapture step may be used to recapture any small molecule analyte which has diffused into the organic (oil) phase during the binding assay.

6.2. Examples

A set of known drugs with different structures and functionalities can be used as test subjects to evaluate methods of retaining a hydrophobic small molecule in an aqueous phase in oil (Pallicer, J. M., et al., ADMET & DMPK (2014) 2(2): 107-114, which is incorporated herein by reference in its entirety). Examples of small molecules that may be used as test subjects include: Sulfanilamide, Acetazolamide, Benzenesulfonamide, Furosemide, Ibuprofen, Clofazimine, and Flufenamic acid. These small molecules range in octanol-water partitioning coefficients (logKow), a measure of expressing the hydrophobicity of a compound, i.e., Sulfanilamide, −0.62 (Hansch, C., Leo, A., D. Hoekman. Exploring QSAR—Hydrophobic, Electronic, and Steric Constants. Washington, DC: American Chemical Society., 1995., p. 22); Acetazolamide, −0.26 (Hansch, C., Leo, A., D. Hoekman. Exploring QSAR—Hydrophobic, Electronic, and Steric Constants. Washington, DC: American Chemical Society., 1995., p. 9); Benzenesulfonamide, 0.31; Furosemide, 2.03; Ibuprofen, 3.97 (Pallicer, J. M. et al., ADMET & DMPK 2(2) (2014) 107-114); Clofazimine, 6.3 (Pallicer, J. M. et al., ADMET & DMPK 2(2) (2014) 107-114); Flufenamic acid, 5.19 (Pallicer, J. M. et al., ADMET & DMPK 2(2) (2014) 107-114).

Oil systems based on perfluorinated oils and perfluorinated surfactants may be used to retain a hydrophobic small molecule in an aqueous droplet within the oil filled gap of a microfluidic device.

To test the effect of different perfluorinated oil and perfluorinated surfactant combinations on small molecule retention in an aqueous phase in oil, an on-bench assay can be used. In one example, the on-bench assay is a modified form of a rolling ball-in-oil liquid-liquid (rbLLE) extraction protocol. The assay uses a surfactant saturated oil mixture that is prepared by mixing an appropriate concentration of perfluorinated surfactant into a perfluorinated oil to achieve the desired interfacial energy for electrowetting. The assay also uses 10 mM solutions of small molecules of interest prepared in an aqueous buffer (PBS+0.1% Tween® 20).

The assay steps are as follows:
Prepare a mixture of perfluorinated surfactant in perfluorinated oil of an appropriate concentration to achieve the desired interfacial energy for electrowetting;
Pipette an aliquot (1 mL) of each diluted saturated surfactant in oil mixture into a separate well in an untreated flat-bottomed 24 well plate (one 24-well plate for each small molecule to be tested);
Pipette an aliquot (1004) of a small molecule solution into each separate well containing an oil/surfactant mixture (i.e., one small molecule solution per plate) and cover the plate with a lid to avoid evaporation and/or cross contamination;
Affix the well plates to an orbital shaker and shake for 15 minutes at 300 rpm;
Extract the aqueous droplet (using a micropipette) from each well into separate labeled Eppendorf tubes and remove as much of the oil phase as possible;
Centrifuge the Eppendorf tubes at 10000 rpm for 3 minutes;
Transfer 75 μL of the aqueous phase from each Eppendorf tube into a separate well of a clean UV transparent flat-bottomed half-volume 96 well plate (replace plate lid between transfers to minimize evaporation);
Transfer 75 μL of each prepared small molecule stock solution (control samples) into separate empty wells of the UV transparent flat-bottomed half-volume 96 well plate;
Place the UV transparent flat-bottomed half-volume 96 well plate into an HTX Synergy Multiplate UV-Vis Reader and obtain absorbance spectra for all wells;
Perform data analysis to compare the absorbance of the control against the sample subjected to the rolling ball-in-oil liquid-liquid (rbLLE) extraction.

Perfluorinated oil and perfluorinated surfactant mixtures tested are shown in Table 1.

TABLE 1
Perfluorinated oil and surfactant mixtures

| Mixture | Perfluorinated oil | Perfluorinated surfactant |
|---|---|---|
| 1 | GALDEN ® (Solvay) HT-170 | Capstone ™ FS-3100 |
| 2 | GALDEN ® (Solvay) HT-170 | Capstone ™ FS-22 |
| 3 | GALDEN ® (Solvay) HT-55 | Capstone ™ FS-3100 |
| 4 | GALDEN ® (Solvay) HT-55 | Capstone ™ FS-22 |
| 5 | 3M ™ Novec ™ 7500 | Capstone ™ FS-3100 |
| 6 | 3M ™ Novec ™ 7500 | Capstone ™ FS-22 |
| 7 | 3M ™ Novec ™ HFE-7200 | Capstone ™ FS-3100 |
| 8 | 3M ™ Novec ™ HFE-7200 | Capstone ™ FS-22 |
| 9 | 3M ™ FC-770 | Capstone ™ FS-3100 |
| 10 | 3M ™ FC-770 | Capstone ™ FS-22 |
| 11 | PDMS (control) | Tween ® 80 (control) |

6.2.1. Cyclodextrin Capture and Release

A cyclodextrin capture and release system may be used to retain a hydrophobic small molecule in an aqueous droplet within the oil filled gap of a microfluidic device. The hydrophobic small molecule can then be released from the cyclodextrin complex at a point-of-assay (e.g., a sensor or detector site).

In one aspect, a cyclodextrin capture and release system uses a displacement mechanism to release the small molecule from a cyclodextrin complex. As described herein above with reference to FIG. 2, a hydrophobic small molecule guest in a cyclodextrin complex can be released via displacement. In the example shown in FIG. 2, the cyclodextrin-small molecule complex (CD-SM) is in an aqueous phase topped by an organic (oil) phase. The CD-SM complex retains the hydrophobic small molecule in the aqueous phase and may be used to substantially reduce partitioning of the hydrophobic small molecule into the oil phase. The small molecule guest in the CD-SM complex is released by the addition of a displacing guest molecule. Upon release from the cyclodextrin complex, the small molecule is free to diffuse from the aqueous phase into the organic (oil) phase.

In one aspect, a cyclodextrin capture and release system uses a light-triggered mechanism to release a small molecule from a cyclodextrin complex at the point-of-assay (e.g., at a sensor or detector site).

To test the capture and retention of a hydrophobic small molecule in a cyclodextrin complex in an aqueous environment surrounded by an oil phase, an on-bench assay can be used. In one example, the assay uses β-cyclodextrin as the cyclodextrin host and PDMS+0.01% Span 85 as the oil phase. Examples of small molecule guests include: Sulfanilamide, Benzenesulfonamide, Furosemide, Ibuprofen, Clofazimine, and Flufenamic acid.

The capture and retention assay steps are as follows:
Prepare a 10 mM solution of each small molecule (SM) of interest in PBS+0.1% Tween® 20;
Prepare 200, 100, 50, and 10 mM solutions of β-cyclodextrin in PBS+0.1% Tween® 20 (“CD” solution); Separately, combine 0.5 mL of a SM solution with 0.5 mL of each concentration of CD solution and vortex each CD-SM mixture;
Prepare control samples of each small molecule solution and each CD solution: combine 0.5 mL of a small molecule solution with 0.5 mL PBS+0.1% Tween® 20, and combine 0.5 mL of a CD solution with 0.5 mL PBS+0.1% Tween® 20;
Analyze the CD-SM mixtures for CD-SM inclusion complex formation, e.g., using capillary electrophoresis or NMR spectroscopy;
Pipette an aliquot (1 mL) of a PDMS+0.01% Span 85 oil solution into each well of an untreated, flat-bottomed 24 well plate;
Transfer, into separate wells containing PDMS+0.01% Span 85, 100 μL of a CD-SM mixture and cover the plate with a lid;
Secure the well plate on an orbital shaker and shake for 15 minutes at 300 rpm;
Extract the aqueous droplet (using a micropipette) from each well into separate labeled Eppendorf tubes and remove as much of the oil phase as possible;
Centrifuge the Eppendorf tubes at 10000 rpm for 3 minutes; Analyze the CD-SM mixtures for SM retention and compare to control samples.

Analysis of a CD-SM mixture for small molecule retention can be performed, for example, using capillary electrophoresis.

In another example, analysis of a CD-SM mixture for small molecule retention can be performed using nuclear magnetic resonance (NMR) spectroscopy. Accordingly, when preparing solutions for the capture and retention assay, all solutions (i.e., cyclodextrin, small molecule, and control samples) are prepared using D₂O.

A displacing guest molecule may be used to release a small molecule from a cyclodextrin inclusion complex. Examples of displacing guest molecules included: a bolaform surfactant (e.g., C10- or C12-bis(ammonium bromide)), SDS, or adamantane carboxylate. A suitable concentration of a displacing guest molecule for releasing a captured small molecule can be determined using an on-bench assay.

The assay steps may include:
Prepare a displacing guest molecule solution in PBS+0.1% Tween® 20 (e.g., a 200 mM solution);
Prepare 200, 100, 50, and 10 mM solutions of β-cyclodextrin in PBS+0.1% Tween® 20 (“CD” solution);
Prepare a small molecule solution in PBS+0.1% Tween® 20 (e.g., a 10 mM solution);
Combine an 0.5 mL aliquot of each concentration of CD solution and 0.5 mL of the small molecule solution and vortex each CD-small molecule mixture;
Analyze the CD-small molecule mixtures for inclusion complex formation;
Combine an 0.5 mL aliquot of a displacing guest molecule solution with the CD-small molecule mixture;
Analyze the CD-small molecule-displacing guest mixtures for inclusion complex formation.

A light-triggered mechanism may be used to release a small molecule from a cyclodextrin inclusion complex.

6.2.2. Aqueous Phase Surfactants

A surfactant or mixture of surfactants may be used to provide anti-partitioning properties to the aqueous phase of a droplet including a hydrophobic small molecule.

To test the effect of different surfactants on small molecule retention in an aqueous phase in oil, an on-bench assay can be used. In one example, the on-bench assay is a modified form of a rolling ball-in-oil liquid-liquid (rbLLE) extraction protocol. The assay uses an oil mixture including PDMS and 0.01% Span 85 as the oil phase. The assay also uses 10 mM solutions of small molecules of interest prepared in an aqueous PBS buffer. To each small molecule solution to be tested, a surfactant (detergent) from a Detergent Screen™ kit (i.e., containing 96 different surfactants; available from Hampton Research; Aliso Viejo, CA) is added to an aliquot of an aqueous small molecule solution. Most of the surfactants (detergents) provided in the Detergent Screen™ kit is at 10× critical micelle concentration (CMC). In the following assay format, combining an aliquot of a surfactant solution with an aliquot of a small molecule solution results in a final surfactant concentration of about 1.5×CMC.

The assay steps may include:
For each combination of small molecule of interest and surfactant to be tested, combine 170 μL of a small molecule solution with 30 μL of a surfactant (detergent) solution in a microtube (reserve remaining small molecule-surfactant mixtures for control samples);
Pipette an aliquot (1 mL) of the PDMS+0.01% Span 85 oil mixture into each well of an untreated flat-bottom 24 well plate;
For each small molecule/surfactant mixture to be tested, pipette an aliquot (100 μL) of the mixture into separate wells of the 24 well plate containing the PDMS+0.01% Span 85 oil mixture and cover the plate with a lid to avoid evaporation and/or cross-contamination (Note that for 96 surfactants to be tested, 4×24 well plates can be used);
Affix the well plates to an orbital shaker and shake for 15 minutes at 300 rpm;
Extract the aqueous droplet (using a micropipette) from each well into separate labeled microtubes and remove as much of the oil phase as possible;
Centrifuge the microtubes containing the extracted aqueous phase at 10,000 rpm for 3 minutes;
Transfer 75 μL of the extracted aqueous phase from each microtube into separate wells of a clean UV transparent flat-bottomed half-volume 96 well plate and cover with a lid;
Transfer 75 μL of the reserved small molecule-surfactant control mixture from each small molecule-surfactant mixture to be tested into separate wells of a clean UV transparent flat-bottomed half-volume 96 well plate and cover with a lid;
For each 96 well plate, obtain absorbance spectra for all wells (e.g., using a HX Synergy Multiplate UV-Vis Reader instrument);
Perform data analysis to compare the absorbance of the control samples (i.e., the reserved small molecule-surfactant control mixtures) against the samples subjected to the rolling ball-in-oil liquid-liquid (rbLLE) extraction.

The assay may be performed using modified conditions to determine if less of a surfactant solution may be used. In one example, the assay may be performed using 1 mL PDMS+0.01% Span 85 and then adding 100 μL of the aqueous phase. In another example, the assay may be performed using 100 μL of the aqueous phase and then adding 1 mL PDMS+0.01% Span 85. In yet another example, the assay may be performed using 0.5 mL PDMS+0.01% Span 85 and then adding 50 μL of the aqueous phase.

1. A method for assaying a small molecule, the method comprising:
(a) providing a droplet actuator comprising two substrates separated to form a droplet operations gap, the substrates comprising electrodes arranged for conducting droplet operations, the droplet operations gap comprising an oil filler fluid;
(b) loading a capture element-small molecule complex aqueous sample onto the droplet actuator, the capture element-small molecule complex comprising a capture element and a small molecule forming an inclusion complex;
(c) dispensing one or more sample droplets from the aqueous sample, wherein dispensing is accomplished using droplet operations, such as electrowetting-mediated droplet operations;
(d) optionally, diluting the one or more sample droplets one or more times by using droplet operations to merge the sample droplet with one or more droplets comprising a dilution buffer;
(e) transporting one of the sample droplets of step (c) to an assay sensor zone, wherein the transporting is accomplished using droplet operations, such as electrowetting-mediated droplet operations;
(f) releasing the small molecule from the capture element-small molecule complex;
(g) conducting a binding assay using the released small molecule; and
(h) measuring binding kinetics of the released small molecule to a ligand.

2. (canceled)

3. (canceled)

4. The method of claim 1, wherein the capture element is selected from the group consisting of a cyclodextrin, a β-cyclodextrin, carrier bead, liposome, Metal Organic Framework (MOF), metallacage (metal-based supramolecular coordination cage), helicate construct, polymer network, DNA-based construct, and gold-nanoparticle.

5. The method of claim 1, wherein the releasing comprises application of light.

6. The method of claim 5, wherein the light is provided via a sensor optical guide.

7. The method of claim 1, wherein the capture element is the cyclodextrin or the β-cyclodextrin and the releasing comprises introduction of a displacing guest molecule agent.

8. The method of claim 7, wherein the displacing guest molecule agent is selected from the group consisting of adamantane carboxylate, SDS, a bolaform surfactant, C10-bis(ammonium bromide), C12-bis(ammonium bromide), and combinations thereof.

9. The method of claim 1, wherein the capture element is the cyclodextrin or the β-cyclodextrin and the releasing comprises application of light.

10. The method of claim 1, wherein the capture element is a liposome and the releasing comprises application of light.

11. The method of claim 1, wherein the capture element is selected from the group consisting of MOF, polymer network, DNA-based construct and gold-nanoparticle and the releasing comprises application of light.

12. The method of claim 1, wherein the releasing comprises a chemistry-based guest release triggering mechanism.

13. The method of claim 12, wherein the chemistry-based guest release triggering mechanism comprises tetrazine-cyclooctene.

14. The method of claim 1, wherein the capture element comprises a carrier bead used to anchor and transport the small molecule.

15. The method of claim 14, wherein the small molecule is anchored to the carrier bead via a cleavable moiety.

16. The method of claim 15, wherein the cleavable moiety comprises a carboxy group.

17. The method of claim 14, wherein the carrier bead is a magnetically responsive bead.

18. The method of claim 177, wherein the carrier bead comprises a magnetic core and a porous outer shell, wherein the small molecule is adsorbed into the outer shell, and wherein the releasing comprises an applied alternating magnetic field.

19. The method of claim 144, wherein the carrier bead is suspendable in the aqueous sample.

20. The method of claim 144, wherein the carrier bead is an agarose bead.

21. The method of claim 144, wherein the carrier bead comprises a functionalization for binding the small molecule.

22. The method of claim 211, wherein the functionalization comprises the cyclodextrin or the β-cyclodextrin.

23.-73. (canceled)
